(19) United States
(12) Patent Application Publication
(10) Pub. No.: US 2008/0114215 A1
(43) Pub. Date: May 15, 2008

(54) SHAPE RECOGNITION OF HYPOGLYCEMIA AND HYPERGLYCEMIA

(75) Inventors: William Kenneth Ward, Portland, OR (US); Robert Bruce, Portland, OR (US)

Correspondence Address: SCHWABE, WILLIAMSON & WYATT, P.C.;PACWEST CENTER, SUITE 1900, 1211 SW FIFTH AVENUE, PORTLAND, OR, 97204, US

(73) Assignee: iSense Corporation, Portland, OR (US)

(21) Appl. No.: 11/558,399

(22) Filed: Nov. 9, 2006

(51) Int. Cl. A61B 5/00

(52) U.S. Cl. 600/300

(57) ABSTRACT

Embodiments of the present invention teach the use of shape recognition methods, apparatuses, and systems to classify the shape of one or more recent glucose trends during continuous glucose monitoring and to warn the user when specific shapes or trends are identified. Using such embodiments, patients with diabetes may be warned before they become overtly hypoglycemic or hyperglycemic.

### TECHNICAL FIELD

Embodiments of the invention relate generally to the field of medical devices and, specifically, to methods, apparatuses, and systems associated with detecting, analyzing, and/or displaying glucose level changes in a body.

### BACKGROUND

In persons with diabetes who take insulin or oral agents, hypoglycemia (low blood sugar) may be a serious event. In some situations, hypoglycemia may lead to loss of cognitive abilities, seizures, stupor or coma. The range of ill effects from hypoglycemia range from embarrassment (losing one's train of thought in a meeting) to more serious outcomes such as auto accidents.

For these reasons, detection of hypoglycemia is one of the most important benefits of continuous glucose sensing. In the most simple case, one can set the level at which the user is alerted to a “threshold” level, for example 65 mg/dl. In this case, whenever the sensed value falls to 65 or below, the alarm is activated.

The problem with such an approach is that when glucose is allowed to fall all the way to the threshold, it may create discomfort for the user (tremor, anxiety, rapid heart rate, sweating). In addition, some patients report problems with cognition even when glucose is only very slightly hypoglycemic (e.g. below 75 mg/dl). Therefore, it is preferable to warn the user before the glucose falls.

Hyperglycemia (elevated blood sugar) may cause problems as well, such as damage to nerves, blood vessels, and organs, and may lead to further serious conditions such as ketoacidosis or hyperosmolar syndrome.

### BRIEF DESCRIPTION OF THE DRAWINGS

Embodiments of the present invention will be readily understood by the following detailed description in conjunction with the accompanying drawings. Embodiments of the invention are illustrated by way of example and not by way of limitation in the figures of the accompanying drawings.

FIGS. 1A, 1B, 1C, and 1D illustrate parabolic curves of glucose versus time data that are concave (FIG. 1A), convex (FIG. 1B) and that are made up from part of the right half (FIG. 1C) or part of the left half (FIG. 1D) of larger parabolas in accordance with various embodiments of the present invention;

FIG. 2 illustrates part of a concave parabola for a glucose versus time curve in accordance with various embodiments of the present invention;

FIG. 3 illustrates part of a concave parabola for a glucose versus time curve in accordance with various embodiments of the present invention;

FIG. 4 illustrates part of a convex parabola for a glucose versus time curve in accordance with various embodiments of the present invention;

FIG. 5 illustrates part of a convex parabola for a glucose versus time curve in accordance with various embodiments of the present invention;

FIG. 6 illustrates part of a convex parabola for a glucose versus time curve in accordance with various embodiments of the present invention;

FIGS. 7A, 7B, 7C, and 7D illustrate risks of hypoglycemia and hyperglycemia for various trends in accordance with various embodiments of the present invention;

FIG. 8 illustrates an exemplary electronic monitoring unit showing various notification and display features in accordance with various embodiments of the present invention; and

FIG. 9 illustrates a comparison of two functions over a defined time period in accordance with various embodiments of the present invention.

### DETAILED DESCRIPTION OF EMBODIMENTS OF THE INVENTION

In the following detailed description, reference is made to the accompanying drawings which form a part hereof and in which is shown by way of illustration embodiments in which the invention may be practiced. It is to be understood that other embodiments may be utilized and structural or logical changes may be made without departing from the scope of the present invention. Therefore, the following detailed description is not to be taken in a limiting sense, and the scope of embodiments in accordance with the present invention is defined by the appended claims and their equivalents.

Various operations may be described as multiple discrete operations in turn, in a manner that may be helpful in understanding embodiments of the present invention; however, the order of description should not be construed to imply that these operations are order dependent.

The description may use perspective-based descriptions such as up/down, back/front, and top/bottom. Such descriptions are merely used to facilitate the discussion and are not intended to restrict the application of embodiments of the present invention.

The description may use the phrases “in an embodiment,” or “in embodiments,” which may each refer to one or more of the same or different embodiments. Furthermore, the terms “comprising,” “including,” “having,” and the like, as used with respect to embodiments of the present invention, are synonymous.

A phrase in the form of “A/B” means “A or B.” A phrase in the form “A and/or B” means “(A), (B), or (A and B).” A phrase in the form “at least one of A, B and C” means “(A), (B), (C), (A and B), (A and C), (B and C) or (A, B and C).” For the purposes of the present invention, a phrase in the form “at least one of A, B, and C” means “(A), (B), (C), (A and B), (A and C), (B and C), or (A, B and C).” A phrase in the form “(A) B” means “(B) or (A B),” that is, A is optional.

In various embodiments of the present invention, methods, apparatuses, and systems for detecting, analyzing, and/or displaying glucose level changes in a body and for shape recognition of hypoglycemia and hyperglycemia are provided. In exemplary embodiments of the present invention, a computing system may be endowed with one or more components of the disclosed apparatuses and/or systems and may be employed to perform one or more methods as disclosed herein.

In various embodiments, the present invention teaches the use of a shape recognition method and apparatus to classify the shape of one or more recent glucose trends during continuous glucose monitoring and to warn the user when specific shapes or trends are identified. Using such a method, patients with diabetes may be warned before they become overtly hypoglycemic or hyperglycemic. In the case of hypoglycemia, patients may treat themselves with rapidly acting carbohydrates in order to prevent frank hypoglycemia. In the case of hyperglycemia, patients may resort to medication, exercise, or a change in diet to affect the rising blood sugar levels.

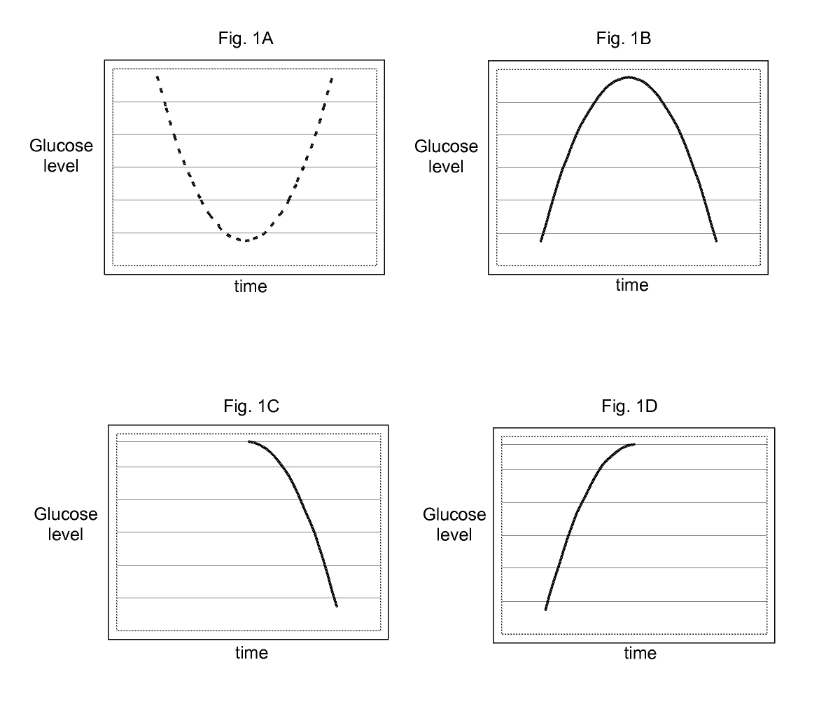

Embodiments of the current invention may be distinguished from the prior art in that neither the slope of a line (rate of change) nor the derivative of the slope (second derivative, acceleration) is utilized to predict future hypoglycemia or hyperglycemia. The rationale for avoidance of the slope calculation is that in animals and humans, the nature of the relationship between glucose and time is rarely that of a straight line. Instead, it is almost always curved. In embodiments, a curve of historical glucose values over a defined period of time may be convex (having the appearance of a hill) or concave (having the appearance of a valley), and may include all or part of a curve. FIGS. 1A, 1B, 1C, and 1D show parabolic curves that are concave (FIG. 1A), convex (FIG. 1B) and that are made up from part of the right half (FIG. 1C) or part of the left half (FIG. 1D) of larger parabolas. It should be understood that, in embodiments, one may use very small parts of a parabolic curve for shape recognition, and such parts may be nearly linear; however, the curvature of the shape of the data points, whether slight or sharp, provides important information about the changing conditions in the body.

When one examines clinical examples of continuous glucose monitoring in patients who take insulin, one may get an understanding of typical shapes of the glucose versus time relationship. For example, one may examine glucose versus time graphs of the 20 minute periods immediately preceding the development in patients of hypoglycemia (defined, for example, as below 65 mg/dl). In these patients, in the time leading up to hypoglycemia, many such shapes may be seen, and rarely is such a shape strictly linear. More likely, the shape is curved upwards (concave) or curved downwards (convex). Quite often, the shape of data over the time period (for example, 20 minutes) preceding hypoglycemia may be fit with a high degree of certainty to a portion of a parabolic curve, such as part of the right side or part of the left side of a parabola.

It should be noted that in the situation where the clinical data do not fit a line, it is not appropriate to characterize the data by the use of a slope of a line (rate of change, velocity). Although one may fit curving data to a line, this creates error and results in loss of some of the integrity of the data. For example, in the situation of a concave data set, one might fit the data to a line (and the correlation coefficient might even be quite high, for example over 0.9). However, if one uses the slope of the line as a means of measuring the rate of decline, one loses the information that characterizes the graph as convex or concave or accounts for such directional change(s).

Thus, in an embodiment of the present invention there is provided a method, wherein the method comprises measuring with a glucose sensing device a plurality of glucose values of an individual for a plurality of points of time over a defined time period; fitting the plurality of glucose values to at least a portion of a curve; identifying a glucose level condition of the individual based on the at least a portion of a curve to which the plurality of glucose values fit; and providing a notification of the glucose level condition to the individual.

For the purposes of various embodiments of the present invention, the term “fitting” refers to the process by which a plurality of data points are fit to a curve in a “best fit” process in which the most suitable curve is approximated from the data provided. In an embodiment, a set of predefined curves may be provided and data points may be fit to one of the predefined curves of the set. In a further embodiment, the curves within the predefined set of curves may be labeled or otherwise provided with a status indicating a glucose level condition or status of the individual from which the data was taken. In other words, various predefined curves may be provided with suitable titles, or glucose level conditions, such as “normal,” “hypoglycemia trend,” “severe hypoglycemia trend” etc. based on the status the data indicates. For the purposes of the present invention, the term “glucose level condition” broadly refers to a past, current, or future identification of a glucose level status.

For the purposes of the present invention, the term “condition of concern” refers to a set of conditions that may be of concern to an individual such as impending hypoglycemia or hyperglycemia. For the purposes of the present invention, the term “impending” refers to a time period that is within a reasonable time in the future such that action may be prudent, such as less than 30-60 minutes.

In an embodiment, measured data points may be fit to a curve and the curve may then be compared to a series of predefined curves that have associated glucose level condition labels such that the measured data points and resultant curve may be provided with the glucose level condition label from the predefined curve to which it most closely matches.

In an embodiment of the present invention, the data fit to curve analysis may be utilized to provide a warning indication if a condition of concern is identified.

In an embodiment of the present invention, there may also be provided a mechanism to adjust the curve-fit analysis and/or the warning indications based on a long term analysis of individualized historical data. In other words, a data set showing the long term glucose values of an individual identifying the regions of concern and the curve shapes that lead to conditions of concern (potentially leading to hypoglycemia or hyperglycemia) may be used to fine-tune a system in accordance with an embodiment of the present invention to recognize those trends in advance and provide a notification to the user.

The following embodiments illustrate examples in which shape recognition may be used to assist in predicting the risk of hypoglycemia or hyperglycemia. For the abbreviations used in these examples, “t” refers to time (in minutes) and “G” refers to glucose level (in mg/dl).

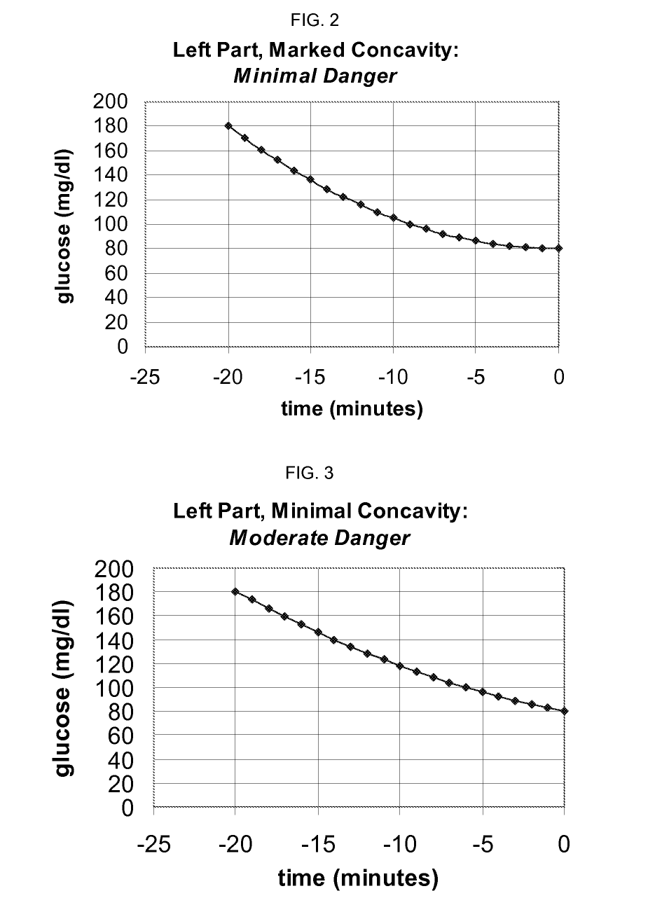

In FIG. 2, the data fit part of the left portion of a concave parabola and the formula for this fit is given as G=80+t̂2/4. In the example of FIG. 2, the glucose value from 20 minutes prior is 180 mg/dl and during the following 10 minutes, the glucose fell extremely quickly. However, as time went on, the glucose level began to fall more slowly. With the understanding that a time of zero is the present time, it may be seen that at the present time, the glucose has essentially stopped its decline and appears to be leveling off. Nonetheless, because of curve-fitting error and noise, it would not be prudent to suggest that there is no risk for hypoglycemia. However, it is important to note that the relative change in curvature over the preceding 20 minutes suggests that the risk of hypoglycemia has shifted. For these reasons, in an embodiment, an individual in this situation may be deemed in minimal danger of developing hypoglycemia.

While an exemplary time period of 20 minutes has been used in various examples herein, it should be appreciated by one of ordinary skill in the art that any suitable time period may be utilized, such as 1, 5, 10, 15, 20, 25, 30 minutes etc. In a functional sense, the time period should be selected such that a sufficient recent history of data may be utilized to provide an indication of the curvature of a graphical representation of the data. The number of data points present for each period of time is dependent upon the sensing system. Thus, in an embodiment, continuous sensing or often-sampled intermittent sensing may provide more accurate data and allow for an accurate curvature to be determined in a shorter duration.

In FIG. 3, similar to that of FIG. 2, the concavity is also part of the left portion of a parabola, but in this case, the parabola is more shallow. This means that the glucose level is not beginning to level off at the present time. Thus, there is a higher risk for hypoglycemia as compared to the situation in FIG. 2. In FIG. 3, G=68+t−10)̂2)/8.

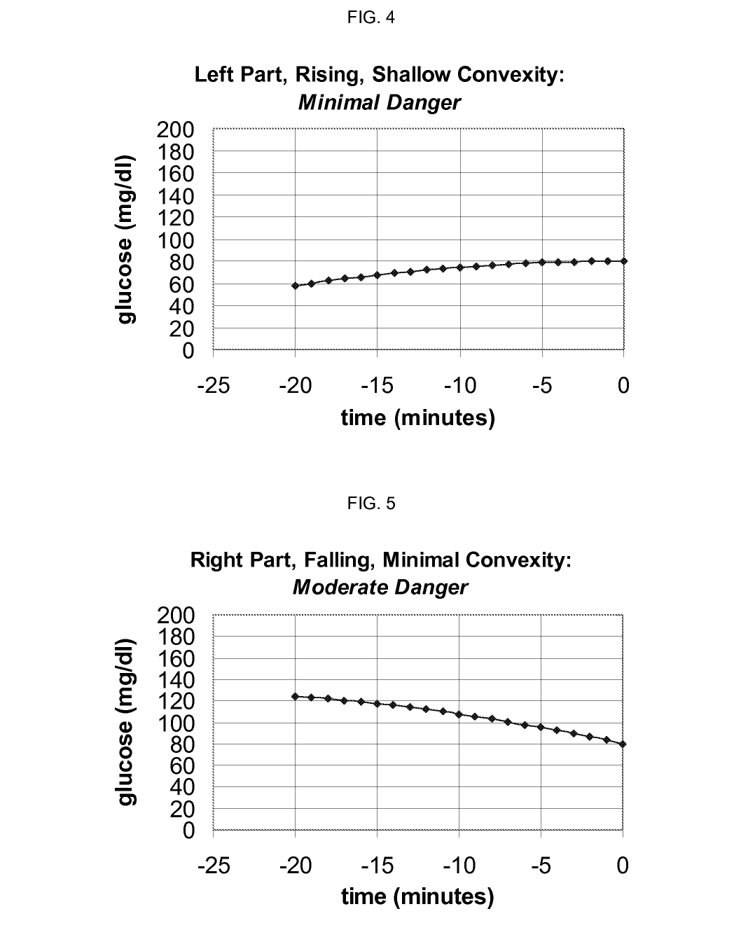

The next three figures illustrate situations in which the shapes are part of convex parabolas. In FIG. 4, the curve is fit to the left part (rising) part of a convex parabola. In other words, the glucose level is rising and has begun to level off at approximately 80 mg/dl. A clinical example of such a situation would be a patient who is recovering from hypoglycemia. In this case, the fact that the glucose is rising suggests a very minimal danger. However the fact that it is leveling off (and not continuing to rise) means that the patient is not entirely free of additional risk for repeat hypoglycemia. In an embodiment, the overall risk for this patient may be considered to be minimal. In FIG. 4, G=80−(t̂2)/18.

FIG. 5 shows a portion of the right part of a convex parabola. In this case, the degree of convexity is minimal and the glucose is falling. In fact, the glucose is falling faster at the present time than it was at any time in the most recent 20 minutes. Given the increasing rate of decline, this shape suggests moderate danger. Therefore, a patient in the situation illustrated in FIG. 5 should be notified (alerted) of the risk of hypoglycemia at a higher glucose level (earlier) than if the degree of curvature were lower. In FIG. 5, G=130−((t+30)̂2)/18.

Thus, as suggested above, in an embodiment of the present invention there is provided a method in which the timing of an alarm (or other notification) is based on the current glucose level and the rate of change in the level. For example, in an embodiment, a faster rate of decline may trigger an alarm earlier than a slower rate of decline, given the same instantaneous glucose level. Such embodiments may be utilized whether the glucose values are fit to a line or to a curve as the rate of change may be determined in either. Although as discussed above, in an embodiment, coupling a determination of rate of decline (or increase) with shape recognition of curves will generally provide a more accurate indication of the current condition (compared to using rate of decline (slope) and data fit to a line).

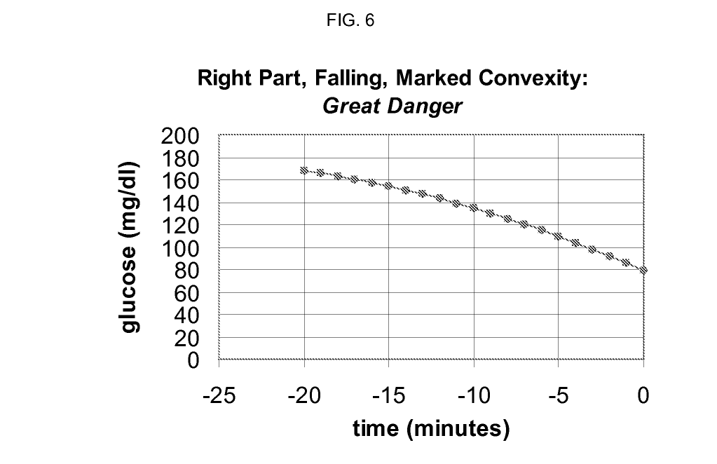

In FIG. 6, the situation is similar to that of FIG. 5 except that the degree of curvature is greater. This means that the glucose level is falling very rapidly at the current time (t=0). From a clinical standpoint, this condition may occur in several situations. One would be the situation in which a patient has hypoglycemia unawareness. In this situation, even when glucose is falling, the body may be unable to secrete hormones that normally inhibit the rate of glucose decline. Such hormones include glucagon, epinephrine, norepinephrine, growth hormone, and cortisol. In such a case, the normal defenses against hypoglycemia are not working. Alternatively, this clinical situation may occur in an individual who has more than one factor that is operating to lower glucose. Such factors may include the combination of recent administration of rapidly-acting insulin and vigorous exercise. In such a case, glucose may fall rapidly and may predispose the patient to very serious hypoglycemia. In FIG. 6, G=179−((t+30)̂2)/9. For the reasons noted above, when the shape of FIG. 6 is recognized, the user needs to be notified at a very early stage.

The following table (Table 1) shows an embodiment of the invention in which different parabolic shapes lead to different degrees of risk, shown in an order from highest risk of hypoglycemia at the top to the lowest risk at the bottom of the table.

TABLE 1
SUMMARY OF SHAPE PATTERNS AND THEIR DEGREE OF RISK

| | | | | Hypoglycemic | Example of glucose level |
|---|---|---|---|---|---|
| | | Portion of | Degree of | trend alert | at which alert should be |
| Direction | Shape | Parabola | curvature | activated? | activated |
| Falling | convex | right | high | yes | 25 mg/dl above threshold |
| Falling | convex | right | medium | yes | 20 mg/dl above threshold |
| Falling | convex | right | low | yes | 15 mg/dl above threshold |
| Falling | concave | left | low | yes | 10 mg/dl above threshold |
| Falling | concave | left | medium | yes | 5 mg/dl above threshold |
| Falling | concave | left | high | no | NA |
| Rising | convex | left | high | no | NA |
| Rising | convex | left | medium | no | NA |
| Rising | convex | left | low | no | NA |
| Rising | concave | right | low | no | NA |
| Rising | concave | right | medium | no | NA |
| Rising | concave | right | high | no | NA |

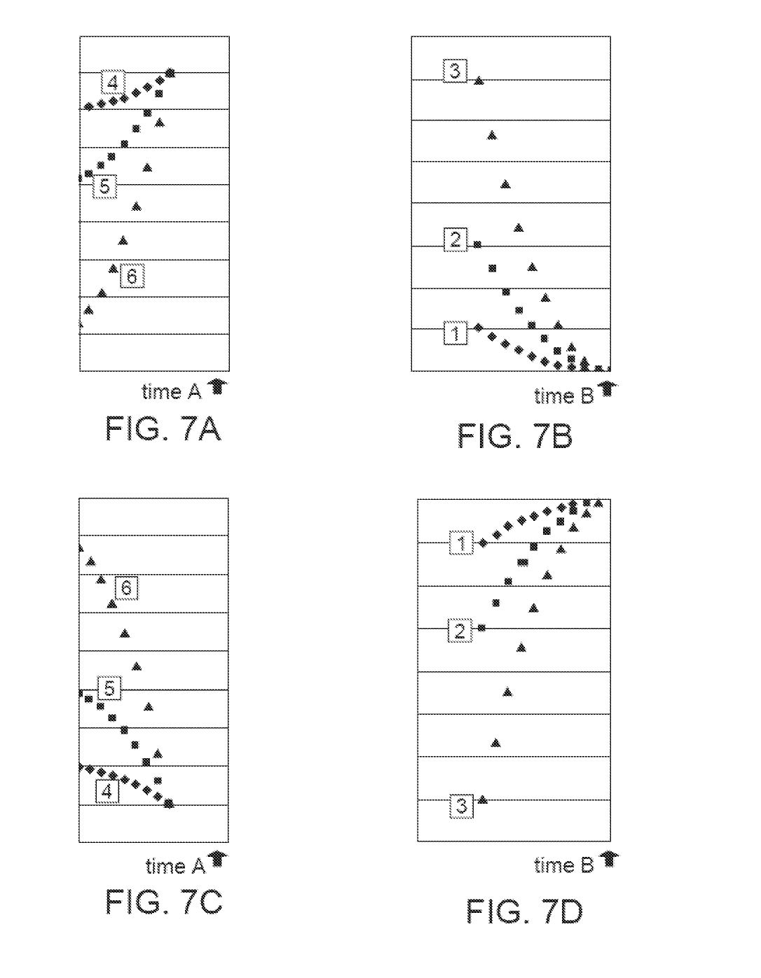

FIGS. 7A, 7B, 7C, and 7D show risks of hypoglycemia and hyperglycemia for various trends of recent glucose values. For each of the figures, the number “6” indicates the greatest risk and the number “1” indicates the lowest risk. As may be seen, in accordance with an embodiment of the present invention, the time at which an individual should be warned is a continuum such that the system provides a warning earlier for a “6” curve (steep) and later for a “1” curve (shallow). FIG. 7A shows the risk of hyperglycemia at time “A” represented by the right sides of three concave parabolas indicated by curves 4 (shallow curve), 5 (medium curve), and 6 (steep curve). FIG. 7B shows the risk of hypoglycemia at time “B” represented by the left sides of three concave parabolas indicated by curves 1 (shallow curve), 2 (medium curve), and 3 (steep curve). FIG. 7C shows the risk of hypoglycemia at time “A” represented by the right sides of three convex parabolas indicated by curves 4 (shallow curve), 5 (medium curve), and 6 (steep curve). FIG. 7D shows the risk of hyperglycemia at time “B” represented by the left sides of three convex parabolas indicated by curves 1 (shallow curve), 2 (medium curve), and 3 (steep curve).

Embodiments of the present invention may be utilized with a variety of known and later developed glucose sensors or monitors. For example, in an embodiment, the glucose sensor may be a small diameter wire-based device that may be inserted under the skin for 3-7 days. In another embodiment, a suitable sensor may be provided in a device that is fully implantable under the skin and that may remain inserted for 3-12 months. The biosensor(s) may be coupled in various ways to implantable or on-skin electrical components and/or external monitoring units that are capable of performing various calculations and analysis and display of data.

In an embodiment, the shape recognized by a sensing/monitoring system, and/or the degree of risk for future hypoglycemia or hyperglycemia, may be displayed on the screen of an electronic monitoring unit that may be, for example, worn on the belt or waistband, or in a table-top unit, to which data may be sent by a wired or wireless connection. In an embodiment, the display may read “Hypoglycemia Trend” or “Hyperglycemia Trend” and at the same time may show a simple graph of the appropriate parabolic shape. In another embodiment, suitable text or a graph may be shown independently.

In embodiments, various types of alarms or notifications may be used to indicate the current condition, especially a condition of concern, such as an audible (alarm or electronic voice prompt), visual (for example colored or flashing lights or a symbol on the display), and/or vibratory notification. In an embodiment, a notification may provide an indication of the degree of risk or the condition of concern. In an embodiment, a notification may also provide an indication or suggestion of an action to be taken as a result of the condition of concern. For example, if it is determined that there is a moderate risk of hypoglycemia developing in the tested individual, the sensing system may provide a suggestion to eat a snack in the next 30-60 minutes. In an embodiment, these suggestions may be customized based on the specific medication, exercise, and dietary parameters of an individual. In another example, if an extreme condition of hyperglycemia is predicted, there may be provided a notification to contact a health care professional to address the situation. In an embodiment, either directly from the sensing device or from a separate monitoring unit, a condition of concern may be communicated further to a medical professional as desired or as programmed into the system, whether communicated manually or automatically.

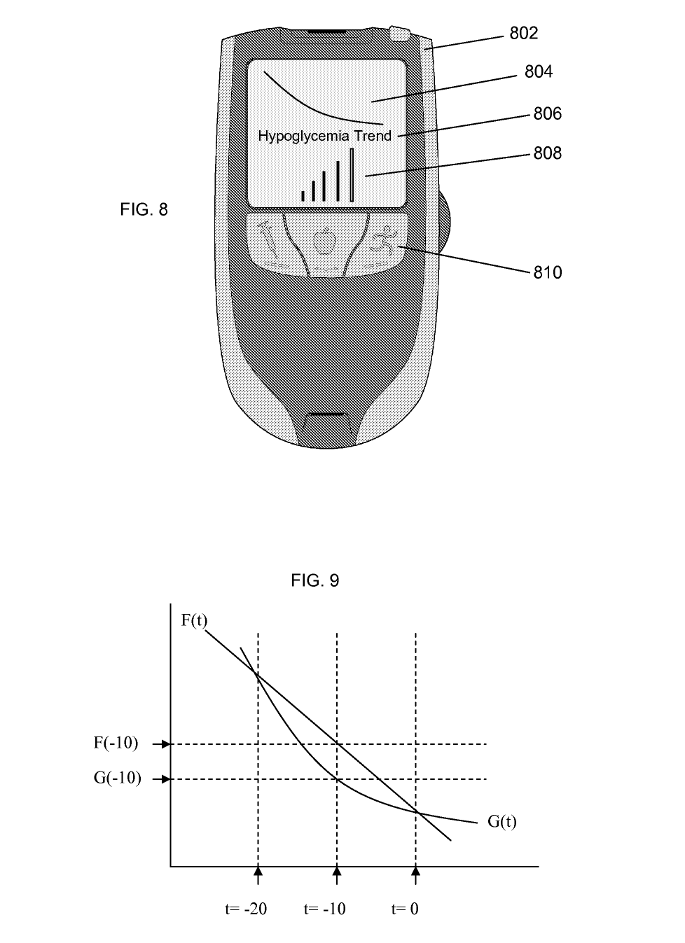

In an embodiment of the present invention as shown in FIG. 8, an exemplary electronic monitoring unit 802 provides various notification and display features. For example, in an embodiment, a graphical representation 804 of a curve of recent historical data may be provided. In addition, or alternatively, in an embodiment, a textual description 806 of the trend may be provided. Various audible or visual displays of the degree of concern may be provided, such as a meter 808, or other lights, flashing or colored (such as a series of green, yellow, and red lights). In addition, electronic monitoring unit 802 may provide an indication of an action to be taken based on the condition or degree of concern using various recommendation buttons or lights 810, providing exemplary recommendation options of an injection, a snack (symbolized by an apple), or exercise. An additional recommendation button may, in an embodiment, provide an indication to contact a medical professional.

For the purposes of understanding the various calculations that may conducted in various embodiments of the present invention, an arbitrary parabola may be defined by the equation y=at²+bt+c (Equation 1), where y is the dependent variable, for example blood glucose in this case, and t is the independent variable, for example time. The coefficients a, b, and c are arbitrary at this point.

A set of N measurements of g at specified times t may be represented as follows: {(t₁,g₁) . . . (t_{N},g_{N})}. Each measurement data point consists of a value for g, a blood glucose value for example, and a value for t, the time when the measurement was taken.

The well-known method of least squares may be used, given a set of data points, to find values for the unknowns a, b, and c in Equation 1 such that Equation 1 approximates the measured data points in that the sum of the squared errors, E², between the measured values of g, given at each data point and the value of g given by Equation 1 may be minimized. The method of least squares is appropriate only if the number of data points is greater than the number of unknowns to be derived. For a parabola, then, using such a method, N must be greater than or equal to four.

The sum of the squared errors may be written as

$${E}^{2} = {\sum }_{i = 1}^{N} {( {g}_{i} - y ( {t}_{i} ) )}^{2} \\ {E}^{2} = {\sum }_{i = 1}^{N} {( {g}_{i} - ( {at}_{i}^{2} + {bt}_{i} + c ) )}^{2} . \qquad ( Equation 2 )$$

In an embodiment, the value of E² may be minimized. It is well known that at the minimum of a function with respect to a variable, the partial derivative of the function is zero. So, taking the partial derivative of Equation 2 with respect to a, b and c and setting them equal to zero gives

$$\frac{\partial {E}^{2}}{\partial a} = 2 {\sum }_{i = 1}^{N} {t}_{i}^{2} [ {g}_{i} - ( {at}_{i}^{2} + {bt}_{i} + c ) ] = 0$$

$$\frac{\partial {E}^{2}}{\partial b} = 2 {\sum }_{i = 1}^{N} {t}_{i} [ {g}_{i} - ( {at}_{i}^{2} + {bt}_{i} + c ) ] = 0$$

$$\frac{\partial {E}^{2}}{\partial c} = 2 {\sum }_{i = 1}^{N} [ {g}_{i} - ( {at}_{i}^{2} + {bt}_{i} + c ) ] = 0$$

Rearranging and expanding the above three equations gives

$${\sum }_{i = 1}^{N} {t}_{i}^{2} {g}_{i} = a {\sum }_{i = 1}^{N} {t}_{i}^{4} + b {\sum }_{i = 1}^{N} {t}_{i}^{3} + c {\sum }_{i = 1}^{N} {t}_{i}^{2}$$

$${\sum }_{i = 1}^{N} {t}_{i} {g}_{i} = a {\sum }_{i = 1}^{N} {t}_{i}^{3} + b {\sum }_{i = 1}^{N} {t}_{i}^{2} + c {\sum }_{i = 1}^{N} {t}_{i}$$

$${\sum }_{i = 1}^{N} {g}_{i} = a {\sum }_{i = 1}^{N} {t}_{i}^{2} + b {\sum }_{i = 1}^{N} {t}_{i} + c {\sum }_{i = 1}^{N} 1$$

The above three equations comprise three linear equations in three unknowns (a, b, and c). There are well-known methods for solving such a system of equations to determine the values of a, b, and c that thus define the parabola of the form of Equation 1 which approximates the data points with minimum sum-squared-error E². See, for example, Morris Hirsch and Stephen Smale, Differential Equations, Dynamical Systems, and Linear Algebra, Academic Press (1974), for a discussion of methods for solving systems of linear equations using, for example, the Gaussian Elimination Method.

In an embodiment, once a, b, and c are found, they may be used to classify the shape of the parabolic curve approximating the data, and to predict the glucose value expected at some time in the near future and/or to provide an indication of the current condition of concern. In an embodiment, a prediction of the future glucose value at a time in the near future (for example, 0-30 minutes) may be made and/or used to determine an appropriate notification or warning to provide to the user.

If the value of a is positive, the parabolic curve is concave. If the value of a is negative, the parabolic curve is convex.

The value of t at the minimum or maximum of the parabola may be found by setting the derivative of Equation 1 equal to zero and solving for t

2at+b=0

2at=−b

t=−b/2a

In an embodiment using the equations above, consider a data interval, for example, covering a period of time from −20 minutes (20 minutes ago) to 0 minutes (the present time). Use the relationship t=−b/2a to find the value of t at the inflection point of the parabola. Now, if t is greater than the maximum time value in the data interval (for example t>0), the inflection point of the parabola lies to the right of the data interval when plotted on the (t, g(t)) plane. Thus, the data interval lies on the “left” side of the parabola. Similarly, if the value of t=−b/2a is less than the minimum value of t in the data interval (for example t<−20), the inflection point of the parabola lies to the left of the data interval when plotted on the (t, g(t)) plane, and the data interval lies on the “right” side of the parabola. If the value of t=−b/2a lies within the data interval, the value of g(t) within the data interval has stabilized or is changing the direction of its trend.

The “degree of curvature” mentioned previously is largely a function of the value of coefficient a determined above. For example, consider the function G(t) shown in FIG. 9. G(t) is shown over the interval for t=−20 to t=0 and displays a significant “curvature” in that range.

Also shown in FIG. 9 is the function F(t) which defines a straight line between the points G(−20) and G(0). Now consider the value C=|F(−10)−G(−10)| as a metric for the curvature of G(t). Since F(t) defines a straight line, F(−10)=(F(−20)+F(0))/2, also, F(−10)=(G(−20)+G(0))/2, and, G(t)=at²+bt+c

C=|G(−20)/2+G(0)/2−G(−10)|

C=|(a(−20)²+b(−20)+2c)/2−(a(−10)²+b(−10)+c))|

C=|200a−10b+c−100a+10b−c|

C=|100a|

Thus, for a given time interval, in an embodiment, an intuitive metric for “degree of curvature” is directly proportional to the value of a. Additionally, in accordance with embodiments of the present invention, it is clear in viewing FIG. 9 that the degree of curvature is an important factor in an accurate prediction of future concerns.

The following table relates the terms “concave” and “convex” used to describe the shape of the parabola to values of a obtained using methods described above:

| Shape | convex | concave |
|---|---|---|
| | a < 0 | a ≧ 0 |

The following table relates the terms “left” and “right” used to describe the portion of the parabola to the values obtained using methods described above. Assuming, for example, a data interval or region of interest encompassing times t from −20 to 0 minutes:

| Portion of parabola | left | right |
|---|---|---|
| | b/2a < 0 | b/2a > 20 |

The following table relates descriptions of “degree of curvature” to values of a obtained using methods described above for glucose data acquired over an exemplary interval of 20 minutes:

| Degree of | | Medium or | |
|---|---|---|---|
| Curvature | Low or minimal | moderate | High or Marked |
| | \|a\| < 0.075 | 0.075 ≦ \|a\| < 0.15 | 0.15 ≦ \|a\| |

Furthermore, given the historical data as discussed above, an estimate or prediction of the glucose value at some time t in the near future may be determined from the equation G(t)=at²+bt+c. For example, an estimate or prediction of the glucose value 20 minutes in the future (t=20) is given by G(20)=400a+20b+c. Note that glucose estimates for other times in the near future may also be estimated. For example, in an embodiment, an estimate of the glucose value 15 or 30 minutes in the future may be clinically useful values. It should be appreciated by those skilled in the art that accuracy of predicted glucose values may be greater for times in the near future versus times farther away. For example, a predicted glucose value for time t=15 minutes will generally be more accurate than a predicted glucose value for t=60 minutes using methods such as described herein.

In an embodiment, if the predicted value for glucose is below some threshold value, for example 50, 60, or 70 mg/dl, an alarm (or other suitable indicator) may be activated to warn the patient that there is a significant risk that they will experience hypoglycemia in the near future. A similar alarm or indication may be activated for a threshold value approaching hyperglycemia.

Although certain embodiments have been illustrated and described herein for purposes of description of the preferred embodiment, it will be appreciated by those of ordinary skill in the art that a wide variety of alternate and/or equivalent embodiments or implementations calculated to achieve the same purposes may be substituted for the embodiments shown and described without departing from the scope of the present invention. Those with skill in the art will readily appreciate that embodiments in accordance with the present invention may be implemented in a very wide variety of ways. This application is intended to cover any adaptations or variations of the embodiments discussed herein. Therefore, it is manifestly intended that embodiments in accordance with the present invention be limited only by the claims and the equivalents thereof.

What is claimed is:

1. A method, comprising:
measuring with a glucose sensing device a plurality of glucose values of an individual for a plurality of points of time over a defined time period;
fitting the plurality of glucose values to at least a portion of a curve;
identifying a glucose level condition of the individual based on said at least a portion of a curve to which the plurality of glucose values fit; and
providing a notification of the glucose level condition to the individual.

2. The method of claim 1, wherein said at least a portion of a curve comprises at least a portion of a parabola.

3. The method of claim 1, wherein said at least a portion of a curve to which the plurality of glucose values fit is one of a predefined set of curves, and said glucose level condition is predetermined for each curve of said predefined set of curves.

4. The method of claim 1, wherein said time period has a duration of approximately 5-30 minutes.

5. The method of claim 1, wherein said notification comprises an audible, visual, or vibratory alarm.

6. The method of claim 1, wherein said notification comprises a display of a graphical representation of said at least a portion of a curve.

7. The method of claim 1, wherein said notification comprises a display of text indicating a condition of concern.

8. The method of claim 1, wherein said notification comprises an indication of a relative level of concern based on the identified glucose level condition.

9. The method of claim 1, wherein said glucose level condition comprises an indication of a prediction of impending hypoglycemia or hyperglycemia.

10. The method of claim 1, further comprising providing a prediction of a future glucose value of the individual for a future point in time based on an extrapolation of the curve to the future point in time.

11. The method of claim 10, wherein said prediction of a future glucose value is displayed on said glucose sensing device or a device associated with said glucose sensing device.

12. The method of claim 10, wherein said prediction of a future glucose value is utilized to determine and provide to the individual a recommended action.

13. The method of claim 12, wherein said recommended action comprises recommending that the individual take a dose of insulin, eat, drink, exercise, and/or contact a medical professional.

14. The method of claim 10, wherein said notification is provided only when the predicted future glucose level of the individual is above or below an established threshold glucose level.

15. The method of claim 14, wherein said threshold is set at or below 70 mg/dl.

16. The method of claim 14, wherein said threshold is set at or above 126 mg/dl.

17. The method of claim 10, wherein said notification is provided when the predicted future glucose level is below an established threshold glucose level and the current glucose level is approximately 5-25 mg/dl above the established threshold glucose level.

18. The method of claim 10, wherein said notification is provided when the predicted future glucose level is above an established threshold glucose level and the current glucose level is approximately 5-25 mg/dl below the established threshold glucose level.

19. An apparatus, comprising:
a glucose sensing device coupled to an electronic monitoring unit, said electronic monitoring unit comprising a storage medium and a plurality of programming instructions stored in the storage medium adapted to program an apparatus to enable the apparatus to:
measure with a glucose sensing device a plurality of glucose values of an individual for a plurality of points of time over a defined time period;
fit the plurality of glucose values to at least a portion of a curve;
identify a glucose level condition of the individual based on said at least a portion of a curve to which the plurality of glucose values fit; and
provide a notification of the glucose level condition to the individual.
